(19) United States
(12) Patent Application Publication
(10) Pub. No.: US 2017/0100348 A1
(43) Pub. Date: Apr. 13, 2017

(54) Treatment Regime for Chronic Sinusitis

(71) Applicant: Microbiome Solutions, LLC, Byron Center, MI (US)

(72) Inventor: Simon Wright, West Des Moines, IA (US)

(21) Appl. No.: 15/291,787

(22) Filed: Oct. 12, 2016

Related U.S. Application Data
(60) Provisional application No. 62/240,293, filed on Oct. 12, 2015.

(51) Int. Cl. A61K 31/05; A61K 47/10; A61K 45/06; A61K 9/00

(52) U.S. Cl. CPC A61K 31/05; A61K 9/0043; A61K 47/10; A61K 45/06

(57) ABSTRACT

The present invention discloses an invention comprising a treatment regime for chronic sinusitis. The regime includes destruction of biofilm and prevention of its formation achieved by a series of applications of compositions that comprise phenol, phenolic, or polyphenolic compounds as a contact/topical application to sinus membranes/tissue. The compositions may comprise phenolic or other compounds sourced naturally. Over-the-counter therapeutic use or in-office application or therapy are all possible implementations.

### CROSS-REFERENCE TO RELATED APPLICATIONS

The present application claims the benefit of U.S. Provisional Patent Application Ser. No. 62/240,293, filed Oct. 12, 2015, which is hereby incorporated by reference herein in its entirety, including any figures, tables or drawings.

### BACKGROUND

Chronic sinusitis or chronic rhinosinusitis is a common condition in which the nasal lining and cavities around nasal passages (sinuses) become inflamed and swollen—for at least eight weeks, despite treatment attempts. The effects of chronic rhinosinusitis typically include interference with drainage and mucus to build up. Chronic sinusitis sufferers may have difficulty breathing through nasal passages. The areas around the eyes and face may feel swollen, and throbbing facial pain or a headache is often present. Recurrent acute sinusitis, subacute sinusitis, sinus headache, FESS failure, allergic rhinitis, atrophic rhinitis, nasal septal perforation and chronic ear pressure are characteristic, together, alone, or in various combinations.

Chronic sinusitis may be caused by an infection associated with a viral upper respiratory tract infection which may be the result of environmental or allergic factors, or associated with gastro esophogeal reflux disease (GERD), immunodificiency or asthma. It may be caused by growths in the sinuses (nasal polyps) or by a deviated nasal septum. In some cases, no specific identifiable cause may be evident.

At least two of the following signs and symptoms must be present for a diagnosis of chronic sinusitis:
Drainage of a thick, yellow or greenish discharge from the nose or down the back of the throat
Nasal obstruction or congestion, causing difficulty breathing through the nose
Pain, tenderness and swelling around the sufferer's eyes, cheeks, nose or forehead
Reduced sense of smell and taste

Other signs and symptoms can include: ear pain, aching in the upper jaw and teeth, cough (which may be worse at night), sore throat, bad breath (halitosis), fatigue or irritability, and nausea.

The signs and symptoms of chronic sinusitis are similar to acute sinusitis, except they last longer and often cause more significant fatigue.

A number of treatments have been developed (with varying levels of effectiveness) to help relieve sinusitis symptoms. These include: Saline nasal irrigation, dead sea salts, Over-the-counter pain relievers (e.g. aspirin, acetaminophen (Tylenol, others) or ibuprofen (Advil, Motrin IB)), nasal corticosteroids to prevent and treat inflammation (e.g. fluticasone (Flonase), budesonide (Rhinocort Aqua), triamcinolone (Nasacort AQ), mometasone (Nasonex) and beclomethasone (Beconase AQ)), oral or injected corticosteroids to relieve inflammation from severe sinusitis (e.g. prednisone and methylprednisolone), decongestants which are available both over-the-counter (OTC) and by prescription in several forms including liquids, tablets and nasal sprays (e.g. Sudafed, Actifed, oxymetazoline (Afrin) or delivered via nebulizer or topical irrigation methods. The overuse of these medications may have the negative effect of rebound congestion and, for that reason, need to be prescribed and used carefully.

Antibiotics are sometimes necessary for sinusitis if a bacterial infection develops. However, chronic sinusitis is often caused by something other than bacteria, so antibiotics do not always have an effect or adequate effect. The preferred mode of treatment using antibiotics starts with a culture to determine the bacteria involved, however, many are prescribed without culture. Most often, oral antibiotics are prescribed.

Therapeutic regimens include the combination of a penicillin such as amoxicillin plus a beta-lactamase inhibitor, a combination of metronidazole plus a macrolide or a second or third generation cephalosporin, and the new quinolones. Where aerobic gram negative organisms are involved, parenteral therapy with an aminoglycoside, 4^{th} generation cephalosporin or fluoroquinolone may be added. Coverage for MRSA is often also included.

In cases that continue to resist treatment or medication, endoscopic sinus surgery may be an option. Depending on the source of obstruction, various instruments may be used to remove tissue or shave away polyps. Enlarging a narrow sinus opening also may be an option to promote drainage.

The goals of medical therapy for CRS are to reduce mucosal edema, promote sinus drainage, and eliminate current infection. Typically, achieving these goals requires a combination of approaches that may include topical or oral glucocorticoids, antibiotics, and nasal saline irrigations. And, although the role of bacteria in the pathogenesis of chronic sinusitis is not completely clear, early combination intensive treatment with oral antibiotics, topical steroids, decongestants and saline nasal sprays often provides symptom relief. If these treatments are not effective, a patient may be referred for surgical evaluation. There is a 25% failure rate of these regimes.

Since the 1980's, “chemical cautery” has been employed to address CRS with varying degrees of success. Chemical cautery comprises in-office application of topical compounds in the sino-nasal tract. These compounds may include silver nitrate, trichloroacetic acid, and disodium chromoglycate. It was generally believed that the treatment acted as a bactericide, killing the bacteria causing the chronic infection. This treatment has not been researched, though clinical experience has suggested potential for certain methods of “chemical cautery.” The rapid development of endoscopic sinus surgical techniques largely eclipsed research interest in this treatment method.

Because of the prevalence of chronic rhinosinusitis and its effects on its sufferers, potentially more effective methods of delivering medications to the sinuses continue to be the subject of studies and research.

Bacteria are culprits in chronic rhinosinusitis and are known to form biofilm in many conditions. A biofilm is considered any group of microorganisms that stick to each other on a surface by association with or embedded in a matrix of extracellular polymeric substance (EPS) better known as “slime.” This slime is a polymeric conglomeration generally composed of extracellular DNA, proteins and polysaccharides. Biofilms may form on living or non-living surfaces. The microbial cells growing in a biofilm are physiologically distinct from planktonic cells of the same organism, which, by contrast, are single-cells that may float or swim in a liquid medium.

Microbes form a biofilm in response to many factors, which may include cellular recognition of specific or non-specific attachment sites on a surface, nutritional cues, or in some cases, by exposure of planktonic cells to sub-inhibitory concentrations of antibiotics. When a cell switches to the biofilm mode of growth, it undergoes a phenotypic shift in behavior in which large suites of genes are differentially regulated.

Antibiotics are not very effective on biofilms even though they may be effective on bacteria which are not incorporated in a biofilm. Previous findings indicate that within the protective environment of the biofilm, the pathogen remains protected from starvation, desiccation and the action of antibiotics. Reports have shown that multidrug resistant strains are also highly efficient biofilm producers, indicating a direct relationship between biofilm formation and antibiotic resistance.

Mechanical cleaning to remove biofilm from non-human surfaces is effective but expensive. Biocides such as chlorine will oxidize and depolymerize the exopolymers in biofilm. Chlorine also diffuses and reacts with the biofilm to loosen and detach it. Hypobromite, ozone, and peroxide are other examples of oxidizing biocides. Nonoxidizing biocides include glutaraldehyde. However, most oxidizing biocides are harmful to other biological tissues, including human tissues such as the nasal tissues.

In light of the issues related to mechanical cleaning and the seeming increase in problems associated with biofilm as it matures, one school of thought is that the likely most effective current approach to biofilm management on biological surfaces is to stop it BEFORE it forms. However, as of the date of this writing, there is actually little known about prevention of formation or about destruction of already formed biofilm on surfaces of a biological host.

Phenol has concentration-dependent effects on tissue and microbial organisms. At concentrations greater than 0.02% it is bacteriostatic. At 0.04-1.0%, it is bacteriocidal. It acts as a fungicide at concentrations greater than 1.3%. At concentrations of 0.5-1.0%, it even has activity against Pseudomonas aerunginosa. (see FIG. 1)

In 2009 the effects of phenol and phenolic compounds on biofilm formation by Pseudomonas aeruginosa were studied (Jagani S, Chelikani R, Kim D S. Effects of phenol and natural phenolic compounds on biofilm formation by Pseudomonas aeruginosa. Biofouling. 2009; 25(4): 321-4.) Here, bacteria and a phenolic compound were incubated together on a plate; the plate was irrigated and then shaken and decanted to remove the bacteria that had not adhered to the plate. The results showed that cell growth per se was not significantly affected by the presence of the phenolic compounds. However, several phenolic compounds showed significant reduction in biofilm formation, i.e., less biofilm formed when certain phenolic compounds were present than formed without the presence of those phenols. Specifically, phenol, polyphenol, Catechin, epigallocatechin gallate, and tannic acid showed significant reduction in formation of biofilm compared to the control. (See FIG. 2)

In 2011, another paper, “Bacterial biofilms and the pathophysiology of chronic rhinosinusitis” was published (Sendamangalam V, Choi O K, Kim D, Seo Y. Biofouling January 2011; 27(1):13-9). This paper confirmed that biofilms are present in CRS and suggested topical treatments for treatment of those biofilms. This paper described that bacteria in the form of a biofilm are resistant to antibiotic therapy and postulated several possible reasons for this effect. Anti-biofilm properties of certain substances were reported. Specifically, tea tree oil (which contains a plethora of terpenes), and manuka honey (which contains hydrogen peroxide, and methylglyoxal) were cited. The paper also theorized that surfactants may lead to disruption of a biofilm as might shear force combined with delivery of a surfactant (however, less than complete treatment of a biofilm and/or further mucosal insult was thought to stimulate proliferation of that bacterial biofilm thereby having an opposite final effect). The paper also suggested interference with quorum sensing might be a reasonable approach to removing or reducing biofilm. The paper theorized that disabling the signaling molecule would weaken the biofilm making it susceptible to other tactics for destruction.

It was an objective of the present invention to provide a means of destroying bacteria on the sinonasal tract for the purpose of decreasing bacterial burden and reducing biofilm formation.

It was an objective of the present invention to provide a means for destroying or reducing biofilm present in a patient suffering from CRS;

It was another objective of the present invention to provide a means for reducing or eliminating re-formation of the biofilm normally associated with CRS;

It was yet another objective of the present invention to provide a therapy for managing or eliminating the symptoms associated with CRS;

It was an objective of the present invention to provide an effective therapeutic means of addressing CRS without surgical treatment;

It was also an objective to identify and develop a treatment regime that is effective across a number of bacterial infections, easily applied, and with lower rates of side effects, complications, and resistance.

### SUMMARY OF THE INVENTION

The present invention comprises a sinus treatment to address acute or chronic sinusitis (sinus infection also referred to herein as CRS). In general, for several years it has been postulated and, more recently, data has been collected that supports a theory that CRS is often associated with the formation of biofilm. The novel treatment functions as a biocide on mucous surfaces with destruction of microbes or stasis of microbes. The novel treatment described herein comprises destruction or reduction of biofilm that is characteristic of the disease. Depending on specific treatment, the novel composition also retards or eliminates re-formation of the biofilm. Specifically, the treatment includes use of a composition that comprises phenol or phenolic compounds as a contact/topical application to sinus membranes/tissue in therapy that comprises sequential treatment with low dose caustic and biocidal agents which may also be used in varied format for chronic maintenance therapy. The phenolic compounds employed may be of natural or synthetic origin. The present invention requires no patient downtime or recovery period; treatment in office or home may be completed in 15 minutes or less; and maintenance applications may be home based. Over-the-counter therapeutic use or in-office application or therapy are all possible implementations.

### DRAWINGS

FIG. 1 is a bar graph showing effectiveness of phenol on Pseudomonas auruginosa

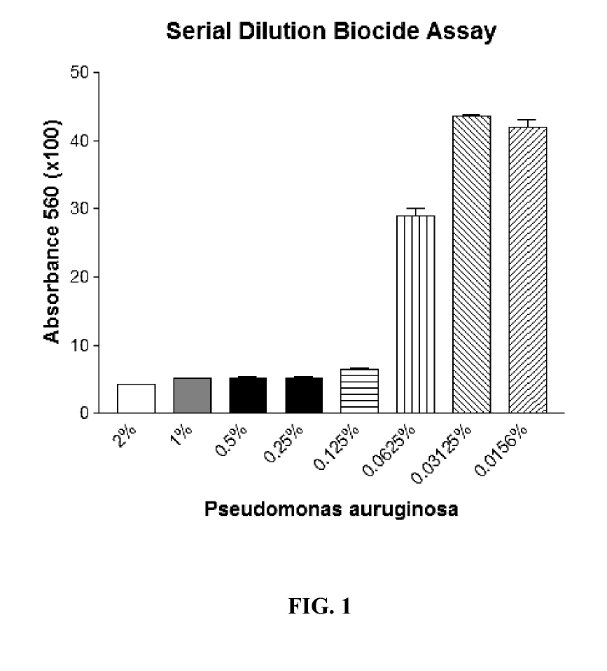

FIG. 2 is a table describing the test group of patients studied

FIG. 3 is a table showing incidence of symptoms in the patient group

FIG. 4 is a bar graph showing effectiveness of phenol on biofilm formation

FIG. 5 is a bar graph showing the SNOT(20) data

FIG. 6 is a bar graph showing Epithelial Indices of CTS FESS histology

FIG. 7 is a bar graph showing Inflammatory Cytology of CTS FESS histology

FIG. 8 is a bar graph showing Stromal Feauter of CTS FESS histology

FIG. 9 is a bar graph showing anti-biofilm properties of phenol and tannic acid

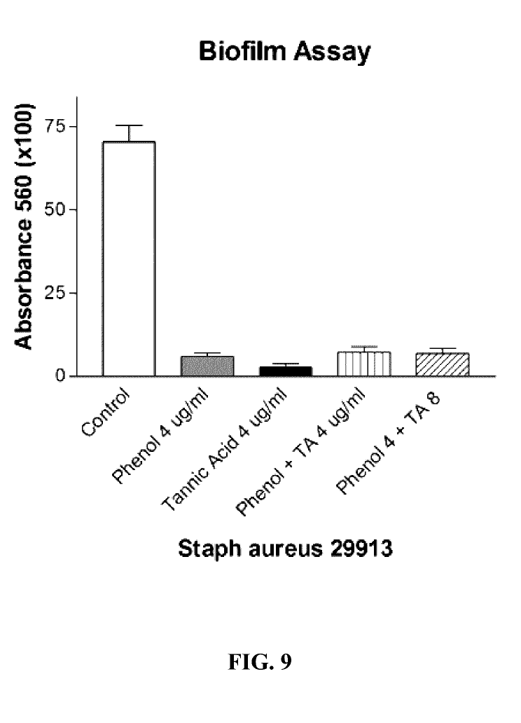

### DETAILED DESCRIPTION

The present treatment regime typically requires a minimum of between 1 and 5 treatments to adequately address CRS. The recommended therapy may comprise several applications relatively close together to eradicate microbes and disrupt the biofilm created by the bacteria associated with CRS and reduce or eliminate the presence of biofilm. These applications may be followed by a maintenance routine that includes application once per month or per two weeks or at another interval that maintains the reduced or eliminated status of the biofilm. Long term more widely-spaced treatments may be given for long term prevention of recurring sinus symptoms in patients predisposed to CRS.

Initial applications of the treatment composition may be of higher dosages accomplished either via higher concentrations, by higher volume, or higher frequency with the maintenance applications perhaps of lessening dose, volume, or spaced temporally further apart or both. The treatment composition may include phenol combined with 1) a substance such as, but not limited to, glycerol or diglycerol or other thickening agent to increase adherence of phenol to the sinus tissue; 2) a substance to assist in lessening the pungent and lasting odor of phenol and/or 3) a substance to increase the comfort of the user such as, but not limited to, glycerin, or Alkalol Nasal Wash which comprises a blend of naturally antiseptic ingredients, including: Purified Water (USP); Menthol; Eucalyptol; Thymol; Camphor; Benzoin; Oils of Wintergreen, Spearmint, Pine, and Cinnamon; Potassium Alum; Potassium Chlorate; Sodium Bicarbonate; Sodium Chloride; Alcohol (2/100 of 1%) and/or 4) a topical decongestant such as oxymetazoline to decongest the nasal passages and allow deeper penetration of the compound, and/or 5) other bactericidal or bacteriostatic agents to augment bacterial eradication. Provided the phenol content does not hinder or destroy its action, other medications or treatment adjuvants can be included in the treatment composition. The phenol or phenolic compound may be naturally sourced from pine or other pine sources or other phenol or phenolic producing plants; or may be sourced industrially where it is produced by controlled chemical reaction. The other components of the treatment composition may also be naturally sourced or GRAS (generally recognized as safe). Further, the treatment may include a recommended sequence of two or three differing compositions over time. The sequence may include consistent phenol dosages or varying phenol dosages. Likewise, the sequence may provide for increases or decreases in a substance to increase adherence of the phenol to sinus tissue or of a substance to increase the comfort of the user. A maintenance routine may also include variations in concentration of phenol over a given time frame in addition to or instead of a change in frequency of application.

Where an in-office treatment is provided, the treatment may begin with application of oxymetazoine (Afrin) and lidocaine followed by a time period adequate to achieve a comfortable level of sensitivity for the patient, typically a few minutes, perhaps about 5 minutes. Thereafter, a sequence of three different solutions is employed: solution 1 comprises a decongestant; solution 2 comprises a cautery agent including any one or more of lugols, phenols, alone or combined; solution 3 comprises a soothing agent. In some applications, solutions 1 and 2 are applied together followed by a short time period, then application of solution 3 or may apply solutions 2 and 3 together. It is recommended that the patient avoid blowing the nose for 2 hours. A treatment plan may comprise treatment once per month for 3months, followed by treatment once every 4 months. This may be varied depending on patient response to the therapy. For example, more resistant cases may retain the once per month a bit longer, or maintenance may be once per 6 weeks or 2 months, etc. At some point in time therapy may cease in order to determine whether the condition has been eradicated and/or to introduce a new routine of treatment. The inventors have determined that a response after the first treatment is a strong indicator that the treatment will be successful. Further, a study conducted by the inventors showed that 90% of patients respond after the third treatment or sooner.

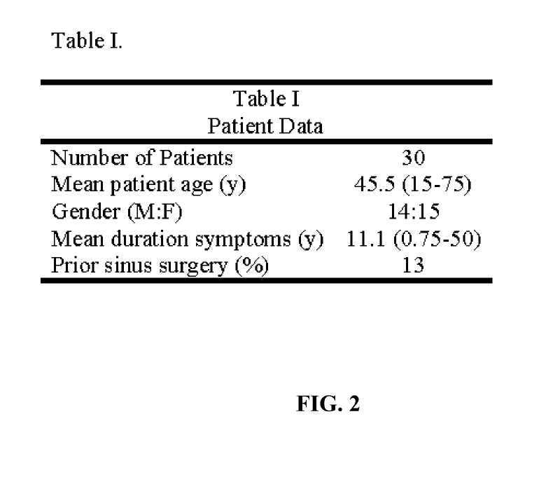

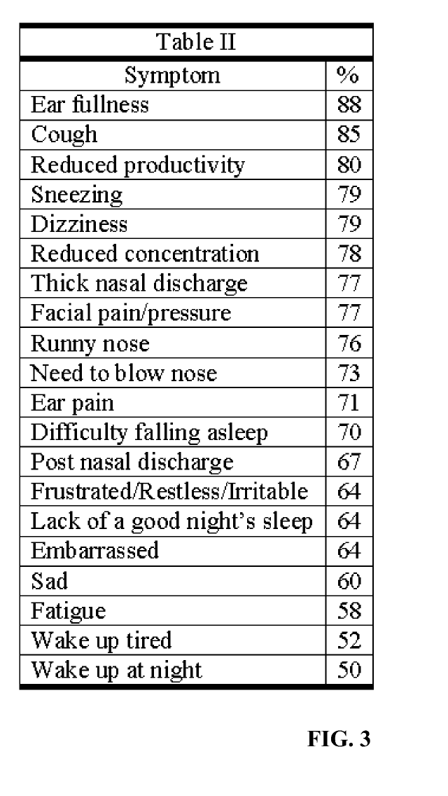

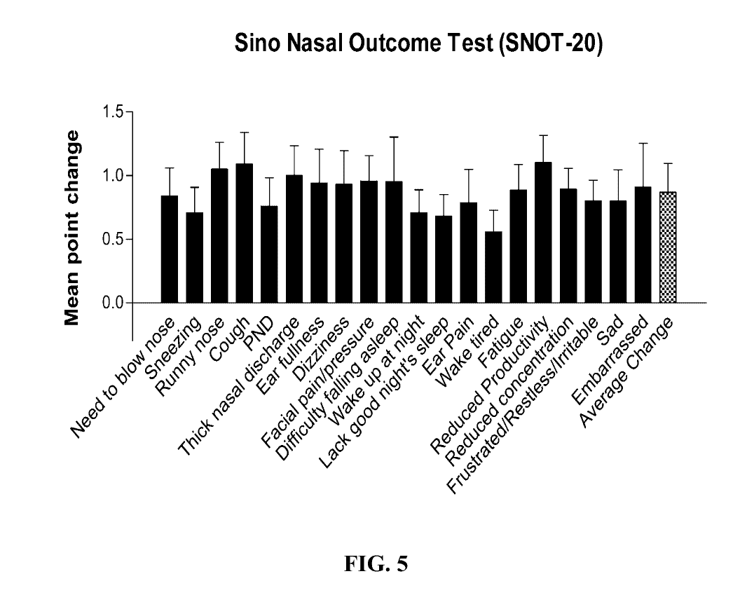

A study of 30 patients was conducted. The subjects reported an average of 11 years having symptoms and had failed multiple prior medical prescriptions before trying the treatment therapy of the prevent invention. Data was collected pre and post treatment and evaluated using RSDI (the Rhinosinusitis Disability Index) and SNOT-20 (Sino-Nasal Outcomes Test) evaluation. The patients underwent one treatment/month, for three months, providing the results shown in FIGS. 2 and 3. The results of the RSDI evaluation showed 100% of patients had relief of some symptoms. In fact, two thirds of the patients reported 80% of their symptoms improved and 9 out of 10 reported that the majority of their symptoms improved. The SNOT-20 evaluation results were also impressive: all patients experienced significant relief and every symptom listed showed improvement. (see FIG. 5)

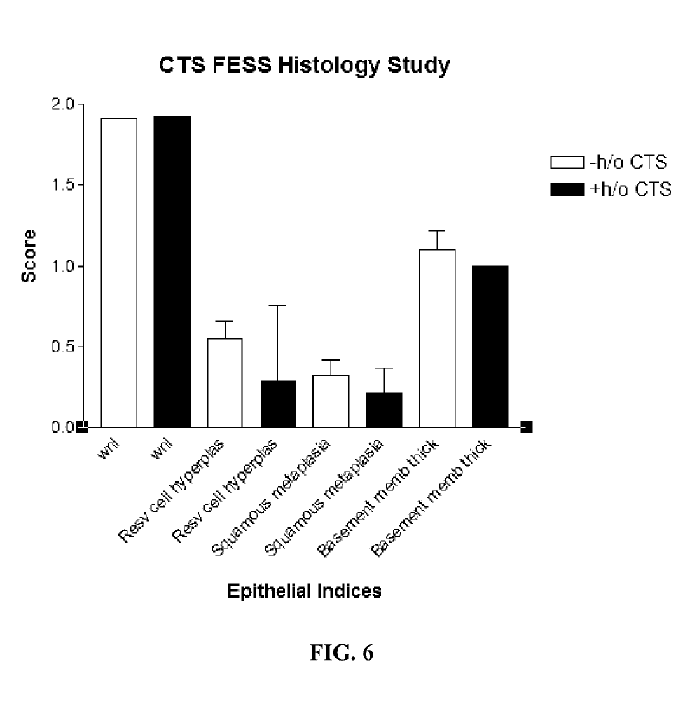

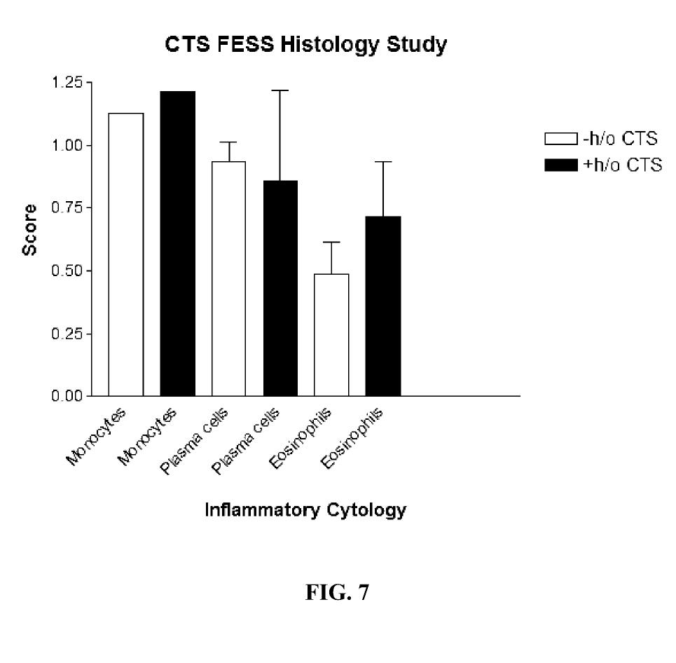

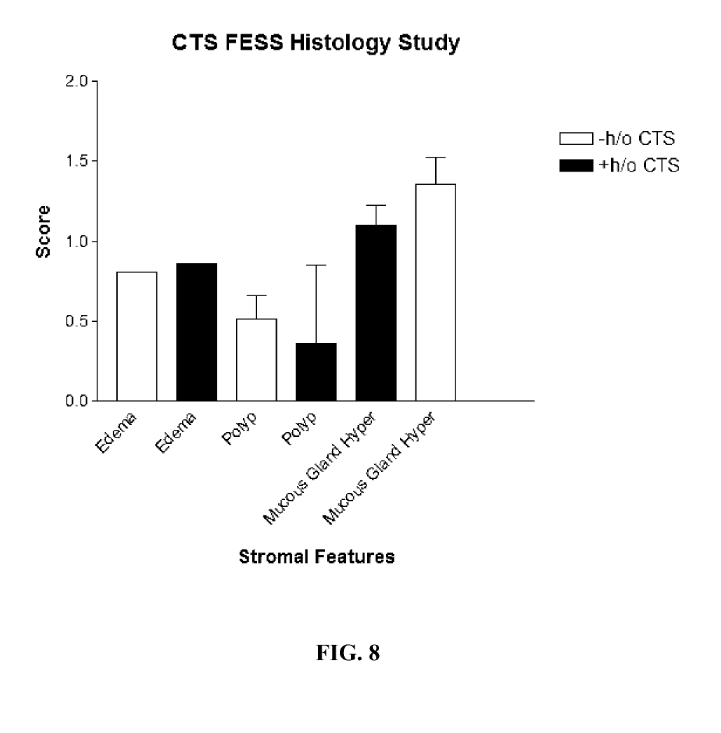

A comparative study was conducted of FESS sinonasal histology of patients that were subjected to the inventive method and those who were not. This study included 14 patients subjected to the method and 31 patients without. Three categories were evaluated: epithelium, inflammation, stroma showing that the scores for those who were subjected to the inventive therapy were lower relative to non-treated by measures of reserve cell hyperplasia, squamous cell metaplasia and basement membrane thickness. Inflammatory study showed higher levels of Monocytes and Eosinophils in those treated, consistent with stabilization of these inflammatory cells. (See FIGS. 6-8)

One embodiment of the present invention comprises an over-the-counter sprayable composition (or compositions) containing phenol, and/or lugols, and/or tannic acid to treat CRS by microbiocidal/microbiostatic effects and removing or disrupting biofilm caused by the bacteria associated with CRS, resulting in changing the microbiome population of the biofilm. The invention may also provide for ongoing therapy to prevent the re-formation of biofilm. Further, the invention covers therapeutic uses of the composition or compositions applied using any of several available therapeutic means including but not limited to irrigation, spray, lotion, etc., in-office or home therapy or a combination of both for the same purpose. Prior art does not teach an over-the-counter or otherwise home application of a phenol-containing composition that includes components to assist in placement and retention of the phenol, lugols and/or tannic acid as well as to provide greater comfort to the user.

Prior art suggests only that phenol may be effective in retarding the formation of biofilm on exposed surfaces but does not teach that phenols destroy already existing biofilm nor does it teach that the microbiome population of a biofilm will be altered by phenol application. Further, prior art makes no suggestion that phenol as a topical application to living organisms will prevent formation of biofilm and, again, makes no suggestion that topical application of phenol to human mucosal cavities would prevent formation or destroy already existing biofilm. The inventor has found no art that teaches that phenol is effective in modulation of mucosal surface biofilm or microbiome composition. The prior art reviewed does not address sinonasal application of phenol or related compounds and is limited to reporting the effects of phenol on formation of biofilm in the setting of a single strain of bacterial culture in petri dish lab environment. It is thought that the present invention destroys biofilm or mediates the microbiome population. It is possible that destruction results from a combination of: a) retarding or halting formation of new biofilm, b) competition within the already present biofilm or environmental factors that cause bacteria to escape the biofilm to recolonize (which possibility is reduced by phenol and/or the body's own immune system which is more effective against bacteria than against biofilm) c) a change in the profile of the microbiome population, d) the body's ability to kill the bacteria that are ejected or escape/leave the biofilm, and e) direct microbiocidal/microbiostatic effects impairing the formation and/or maintenence of biofilms, and f) possible interference with quorum-sensing of bacterial populations.

The present invention includes several possible recommended regimes all of which include the three-fold objectives of a) decreasing the viability of microbial populations of the sinonasal passages, b) disrupting the structure of present biofilm; c) preventing the formation of new biofilm and/or facilitating the body's own defenses to prevent the formation of new biofilm. In some embodiments, an antibiotic or other biocidal agent is simultaneously provided either as part of the phenol-containing composition or as a separate medication. This antimicrobial agent may be specifically selected according to the predominant bacteria present in order to address the bacteria that may be freed from the biofilm or otherwise in the system.

One means to apply the compositions of the present inventive regime contemplates a spray comprising droplets. However, the inventor asserts that various spray characteristics related to size and velocity of droplets may provide the advantages claimed herein and, further, that swab applicators or insertion applicators or even aromatherapy/inhalant type applicators may be applicable and/or effective for some aspects, stages, application of the therapeutic regime of the present invention.

The composition of the present invention comprises Lugol's solution about 1 to about 2%, glycerin 0.25 to about 2%, phenol 0.25-to about 1.0%, and 0.25-0.5% diglycerin or glycerin, and, optionally, any one or more of eucolyptol, menthol, thymol, peppermint oil 0.25-1% which addresses the lingering odor of phenol that is objectionable to some patients. Tannic acid 0.1-2% may be included in the solution. All solutions are in 0.45% saline, however, it is understood that saline is not a critical factor. Phenol is a powerful bacteriocide effective as low as 0.04 to 1.0% and bacteriostatic at 0.03%-0.06% relative to Pseudomonas; it is also fungicidal at concentrations greater than 1.3%. It is believed to have a powerful effect on the formation of biofilm. See Figsl describing its effect on Pseudomonas. It has also been shown to be effective in preventing biofilm formation of streptococcus mutans. Phenols or phenolic compositions employed in the present invention may be naturally sourced from plants, or synthetic, or blends thereof.

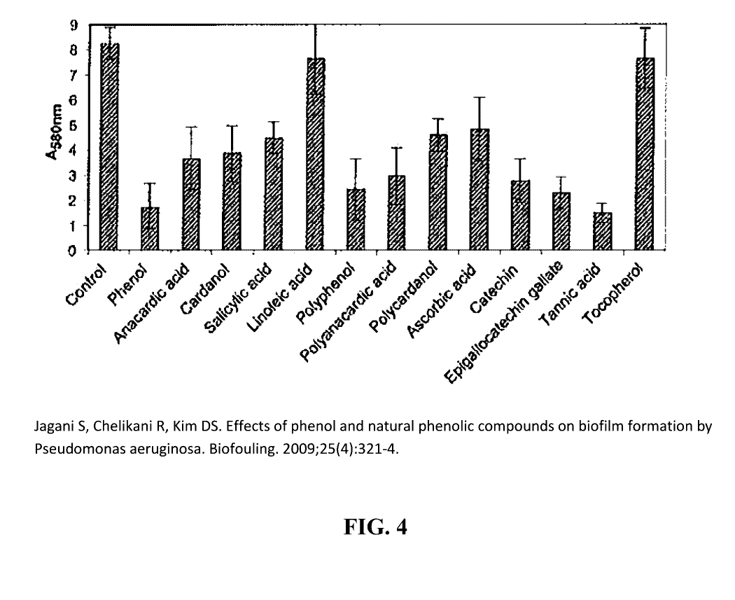

Lugols shows different activity against different microbes; specifically, Lugol's was shown to be biocidal down to 0.125% and bacteriostatic at 0.015% (see FIG. 3) relative to staph aureus and biocidal to 0.125% and bacteriostatic at 0.125% relative to Pseudomonas. Studies also show that phenol and natural phenolic compounds such as tannic acid may affect formation of biofilms at varying degrees of effectiveness. (See FIG. 4).

The treatment regime may include a specific dosage to be sprayed into each nostril. The treatment may comprise a kit having pre-loaded syringes or applicators for the same purpose. Alternatively, a dose or series of doses may be provided preloaded in a bottle, canister, or dosage device. The recommended dosage may be several times per day for several days or weeks, followed by a maintenance routine of variable frequency. In one embodiment, the treatment comprises an initial office visit to administer the primary dose by pressurized atomizer followed thereafter by patient self-administration using a mechanical dosing device such as a syringe, swab, atomizer or other mechanical dosing device. The mechanical dosing device may be pre-loaded with a composition for single use or, alternatively, for multiple use. The mechanical dosing device may each be loaded with one of a plurality of sequential doses which may be identical or may be comprised of active ingredients in accordance with a prescribed sequence. Alternatively, a single mechanical dosing device may be pre-loaded with a plurality of sequential doses that the user simply applies in order. Alternatively, the single mechanical dosing device may be loaded with the components of the composition and provided with means to select the amount of each composition for each dose in accordance with a prescribed regime.

There is no indication that this therapeutic regime is incompatible with other sinus treatment protocols or contra-indicated for such use. Further it is within the scope of the invention to encompass patient self-application of at least the maintenance program or office ENT application. For example, the application may be provided in a pre-measured, single dose applicator of comprising means to administer a series of pre-measured doses. The applicator may retain the components of the regime separately or in mixed form and may or may not comprise components that may be selectively set/adjusted to accommodate for an adjusted prescribed regime or to provide pre-set dosages over the course of the treatment. No recovery time or down time was determined by the inventors to be necessary.

The present invention has been described in certain parameters as means of example. Alternative embodiments may be within the purview of one of ordinary skill in the art upon reading the disclosure herein and are deemed part of the present invention which includes such alternative embodiments within the ordinary skill.

### Outcome Measures and Efficacy of Phenol-Based Topical Sinonasal Treatment . . . Wright, 2004

Effects of phenol and natural phenolic compounds on biofilm formation by Pseudomona aeruginosa Jagani 2009

Bacterial biofilms and the pathophysiology of chronic rhinosinusitis . . . Al-Mutairi 2011
Development of Novel Anti-biofouling Materials from Natural Phenol Compounds, dissertation 2007,
Rahul Chelikani, University of Toledo. Review of portions available on-line. Recommend full review.

What I claim is:

1. A method of treating CRS comprising administering to nasal tissue a composition comprising at least one component and an amount of phenol to destroy biofilm formed by Pathologic sinonasal bacteria.

2. A method of treating CRS comprising administering to nasal tissue a composition comprising at least one component and an amount of phenol to destroy pathologic sinonasal bacteria.

3. The method of claim 2 comprising administering to nasal tissue a series of doses of said composition to prevent the biofilm formation by Pathologic sinonasal bacteria.

4. The method of claim 3 said at least one component comprising at least one antibiotic or biocide to eliminate planktonic bacteria.

5. The method of claim 3 wherein said composition further comprises glycerol.

6. The method of claim 1 wherein a patient self-administers the composition.

7. The method of claim 5 said composition further comprising a component for reducing the characteristic odor of phenol.

8. The method of claim 6 said composition administered in accordance with a dosage plan to deliver specified relative amounts of at least one of the components of the composition.

9. The method of claim 2 said composition further comprising a substance to increase comfort of the nasal tissue, and a topical decongestant.

10. The method of claim 1 wherein the phenol is naturally sourced.

11. The method of claim 3 wherein said series of doses is administered as follows: at least three sequential doses spaced apart by time 1 followed by at least two maintenance doses spaced apart by time 2 which is longer than time 1.

12. The method of claim 11 wherein at least one of said series of doses is administered by the patient.

13. The method of claim 12 wherein said at least three sequential doses spaced apart by time 1 are administered by medical personnel.

14. The method of claim 3 wherein said series of doses may comprises varying concentrations administered over a period of about 3 months or longer.
